(19) United States
(12) Patent Application Publication
(10) Pub. No.: US 2014/0277102 A1
(43) Pub. Date: Sep. 18, 2014

(54) Deep Vein Thrombosis Prevention Garment Having Opposing Offset Attachment Straps

(71) Applicant: Compression Therapy Concepts, Inc., Eatontown, NJ (US)

(72) Inventors: Orlando Mansur, JR., Eatontown, NJ (US); Leonard Nass, Eatontown, NJ (US)

(21) Appl. No.: 14/216,949

(22) Filed: Mar. 17, 2014

Related U.S. Application Data
(60) Provisional application No. 61/786,392, filed on Mar. 15, 2013.

(51) Int. Cl. A61B 17/135

(52) U.S. Cl. CPC A61B 17/135
USPC 606/202

(57) ABSTRACT

A deep vein thrombosis prevention garment includes an S-shaped body having a central panel, a left and a right side panels both formed with a number of offset and opposing attachment straps having an integral fastener. The central panel is formed to have a single air chamber receiving air from a pump through a flexible air supply tube. The central panel, left side panel and right side panel are formed from a single material which does not require a skin-safe liner or other combination of materials. A single-chamber bladder is attached to the central panel. A cover panel, sized to cover the inside surface of the central panel, covers the single-chamber bladder and is attached along its perimeter to the central panel. The cover panel and the body are made from the same material which can be placed against a patient's skin.

### RELATED APPLICATION

This application claims the benefit of priority to U.S. Provisional Application No. 61/786,392, filed on Mar. 15, 2013, entitled “Deep Vein Thrombosis Prevention Garment Having Opposing Offset Attachment Straps”, and currently co-pending.

### FIELD OF THE INVENTION

The present invention relates generally to medical and therapy devices. The present invention is more particularly useful as a compression garment for use in the prevention of deep vein thrombosis. The present invention is particularly useful to prevent deep vein thrombosis during periods of low or no activity by continually circulating blood through a patient's extremities.

### BACKGROUND OF THE INVENTION

Deep Vein Thrombosis, or “DVT”, is a blood clot (“thrombus”) that forms in a vein deep in the body. A thrombus occurs when blood thickens and clumps together. Most of these thrombi occur in the lower leg or thigh; however, they can also occur in other parts of the body. Thrombi located in the thigh are more likely to break off and cause a pulmonary embolism (“PE”) than clots in the lower leg or other parts of the body. The clots that form close to the skin usually cannot break off and cause a PE due to their reduced size and the reduced pressures exerted on them.

A DVT, or a portion of it, can break off and travel through the bloodstream where it can enter the lung and block blood flow. This condition is called pulmonary embolism, which is considered to be very serious due to its likelihood of causing damage to the lungs and other organs and quite possibly leading to death. This condition affects more than 2.5 million Americans each year and is associated with an estimated 50,000 to 200,000 deaths annually.

The venous system is designed to allow for the return of blood to the right side of the heart. Veins are not passive tubes through which blood passes, but are a system that uses muscular compressions, gravity, and inter-venous valves to promote and control the flow of blood through them. The valves are located along the entire length of the vein and ensure that blood only flows in one (1) direction, toward the heart. Blood flow may easily pass through the valve in the direction toward the heart, but when pressure is greater above the valve than below, the cusps will come together, thereby closing the valve and stopping the further flow of blood to the heart.

The valves consist of two (2) very thin-walled cusps that originate at opposite sides of the vein wall and come together to meet at the midline of the vein. The diameter of the vein is slightly larger just behind the valve where the cusps attach to the vein wall. Due to the larger diameter of the vein and the propensity for blood to collect and stagnate between the valve cusps and the vein wall, thrombi formation in this area is more likely.

The most common causes of DVT are venous stasis, blood vessel wall injury, and hypercoaguability. Venous stasis is the reduction of blood flow, most notably in the areas of venous valves, usually caused by extended periods of inactivity. These periods of inactivity minimize the muscular compressions applied to the veins, and therefore remove the forces used to propel the blood through the veins, accordingly. This reduction in flow allows the blood to collect and congeal, thereby forming a clot. The conditions that contribute to venous stasis include heart disease, obesity, dehydration, pregnancy, a debilitated or bed-ridden state, stroke, and surgery. Stasis has been known to develop with surgical procedures lasting as little as 30 minutes.

Vessel wall injury can disrupt the lining of the vein, thereby removing the natural protections against clotting. The loss of natural protection will increase the chances of clot formation and the subsequent mobilization of the clot that can lead to a PE. Some of the major causes of vessel wall injury are trauma from fractures and burns, infection, punctures of the vein, injection of irritant solutions, susceptibility to DVT, and major surgeries.

Hypercoaguability exists when coagulation outpaces fibrinolysis, which is the body's natural mechanism to inhibit clot formation. When this condition exists, the chances of clot formation, especially in areas of low blood flow, are greatly increased. Some causes of hypercoaguability are trauma, surgery, malignancy, and systemic infection. A typical treatment is the administration of an anti-coagulant such as of low-molecular-weight heparin.

It is recognized that clots usually develop first in the calf veins and “grow” in the direction of flow in the vein. The clots usually form behind valve pockets where blood flow is lowest. Once a clot forms, it either enlarges until it is enveloped, which causes the coagulation process to stop, or the clot may develop a “tail” which has a high chance of breaking off and becoming mobile where it can enter the pulmonary system and become lodged in the lungs.

In a patient with DVT, the goals are to minimize the risk of a PE, limit further clots, and facilitate the resolution of existing clots. If a potential clot is suspected or detected, bed rest is usually recommended to allow for the clot to stabilize and adhere to the vein wall, thereby minimizing the chance of the clot becoming mobile such that it can travel to the lungs. A more effective preventative measure is ambulation, which is to walk about or move from place to place. Ambulation requires muscle movement. The muscle movement will provide a continuous series of compressions to the veins, thereby facilitating the flow of blood. The continuous flow of blood will reduce or eliminate any areas of stasis so clots do not have a chance to form. For people who are confined to a bed or will be immobile for an extended period of time, leg elevation is recommended. This will promote blood return to the heart and will decrease any existing venous congestion.

Graduated compression stockings have also been used to apply pressure to the veins so as to reduce or minimize any areas of low flow in the vein, while not allowing the collection and coagulation of blood in these low flow areas. The stockings are designed to provide the highest level of compression to the ankle and calf area, with gradually decreasing pressure continuing up the leg. The stockings prevent DVT by augmenting the velocity of venous return from the legs, thereby reducing venous stasis. Typically, stockings are applied before surgery and are worn until the patient is fully able to move on their own. The stockings need to fit properly and be applied correctly. If too tight, they may exert a tourniquet effect, thereby promoting venous stasis, the very problem they intend to prevent. If too loose, the stockings will not provide adequate compression.

Another treatment of DVT involves the use of intermittent pneumatic compression (IPC). IPC can be of benefit to patients deemed to be at risk of deep vein thrombosis during extended periods of inactivity, and is an accepted treatment method for preventing blood clots or complications of venous stasis in persons after physical trauma, orthopedic surgery, neurosurgery, or in disabled persons who are unable to walk or mobilize effectively.

An IPC uses an air pump to inflate and deflate airtight sleeves wrapped around the leg. The successive inflation and deflations simulate the series of compressions applied to the veins from muscle contractions, thereby limiting any stasis that can lead to thrombi formation. This technique is also used to stop blood clots from developing during surgeries that will last for an extended period of time.

While there are a number of airtight sleeves that have been developed for IPC, the available sleeves are created from multi-layered materials and are relatively expensive to manufacture. For instance, in currently available airtight sleeves, an air bladder is provided and encased in a multi-layered garment that requires a great deal of manufacturing effort, including the careful cutting and stitching of multiple layers of cloth suitable for prolonged placement against a patient's skin. Indeed, the airtight sleeves currently available are formed with a soft inner layer material that is suitable for contact with the skin, and an outer layer that is more durable and serves as a backing to provide the necessary compression to the patient. This two-layer construction results in an expensive and complicated manufacturing of the airtight sleeve. Also, the combination of two (2) dissimilar materials requires a perimetric piping that serves to finish the cut edges of the two (2) dissimilar materials, to connect the two (2) materials together, and to provide a finished edge.

In light of the above, it would be advantageous to provide a deep vein thrombosis prevention garment that minimizes the occurrence of deep vein thrombosis formation. It would be further advantageous to provide a deep vein thrombosis prevention garment that allows medical personnel to customize the compression of limbs being treated to optimize treatments for particular patients. It would be further advantageous to provide a deep vein thrombosis prevention garment that is made from a single panel of material thereby minimizing manufacturing costs. It would also be advantageous to provide a deep vein thrombosis prevention garment that is easy to use, relatively easy to manufacture, and comparatively cost efficient.

### SUMMARY OF THE INVENTION

The deep vein thrombosis prevention garment having opposing offset attachment straps (hereafter referred to as the “deep vein thrombosis prevention garment”) of the present invention includes an S-shaped body having a central panel, a left side panel, and a right side panel, with left and right side panels formed with opposing and offset attachment straps having integral fasteners, such as Velcro® brand hook and loop fasteners. The central panel is formed to have a single air chamber which receives air from a pump through a flexible air supply tube.

The central panel, left side panel, and right side panel are formed from a single material which requires no skin-safe liner or other combination of materials. A single-chamber bladder is attached to the central panel. A cover panel sized to cover the inside surface of the central panel, covers the single-chamber bladder and is attached along its perimeter to the central panel. The cover panel and the body are made from the same material which can be placed against a patient's skin.

In use, the central panel is positioned against a large muscle in the limb being treated, such as the calf muscle, and the left side panel is wrapped in one (1) direction around the limb, with the left side attachment strap attached to the outside surface of the central panel. The offset and opposing right side panel is wrapped around the limb from the other direction and attached to the outside surface of the central panel to secure the device around the patient's limb.

The deep vein thrombosis prevention garment of the present invention is worn by a patient on an extremity that is subject to development of thrombosis, particularly deep vein thrombosis, and particularly during surgery or extended periods of inactivity. In use, the deep vein thrombosis prevention garment is wrapped snugly around a patient's leg, for example. Once activated, the pump provides a periodic air supply to the garment through the flexible air supply tube leading to the air chamber.

The air pressure is maintained through the flexible air supply tube and the air filled chamber becomes pressurized to a predetermined pressure, such as 35 mmHg. As the air filled chamber inflates, it provides additional pressure on the leg of the patient to urge blood flow further upward through the leg.

The inflation of the air filled chamber, coupled with the valves within the venous structure of the limb, creates a peristaltic force on the veins within the limb being treated. Once the air filled chamber is pressurized to a predetermined pressure, the pressurized air supplied to the flexible air supply tube is discontinued, and the air filled chamber deflates, returning the deep vein thrombosis prevention garment of the present invention to its fully un-inflated configuration. In this fully un-inflated configuration, blood flows freely through the limb being treated.

The inflation and deflation timing cycle of the deep vein thrombosis prevention garment of the present invention is determined by the pressures being utilized, and the speed by which the air filled chambers deflate. In order to effectively urge blood flow through deep veins, the timing for the peristaltic effect of the deep vein thrombosis prevention garment of the present invention is approximately twenty (20) seconds per cycle.

### BRIEF DESCRIPTION OF THE DRAWING

The nature, objects, and advantages of the present invention will become more apparent to those skilled in the art after considering the following detailed description in connection with the accompanying drawings, in which like reference numerals designate like parts throughout, and wherein:

FIG. 1 is a top plan view of a preferred embodiment of the deep vein thrombosis prevention garment of the present invention showing a central panel, an offset left side panel and right side panel both formed with an attachment strap having an integral fastener, and with the central panel having an air filled chamber (shown in dashed lines) receiving air from a pump through a flexible air supply tube;

FIG. 2 is a view of the deep vein thrombosis prevention garment of the present invention being used by a patient for the prevention of deep vein thrombosis, showing a pump supplying pressurized air through a flexible air supply tube;

FIG. 3 is a side cross-sectional view of the deep vein thrombosis prevention garment of the present invention as taken along line 3-3 of FIG. 1, showing the relative positions of the air filled chamber when the deep vein thrombosis prevention garment is in a partially inflated configuration, and demonstrating the flow of air from the air supply tube into the air chamber to fill the chamber;

FIGS. 4-5 depict the deep vein thrombosis prevention garment of the present invention as used on the leg of a patient starting with an un-inflated configuration, and advancing through the inflation of the air filled chamber;

FIG. 4 is an exemplary partial cross-sectional view of the deep vein thrombosis prevention garment of the present invention as used on the leg of the patient, showing the deep vein thrombosis prevention garment in a deflated configuration in which little or no pressure is exerted on the leg of the patient and blood flows unrestrictedly through the leg;

FIG. 5 is an exemplary partial cross-sectional view of the deep vein thrombosis prevention garment of the present invention as used on the leg of the patient, showing the deep vein thrombosis prevention garment in a partially inflated configuration with the air filled chamber filled with air to provide pressure on the leg of the patient to urge flow upward through the leg;

FIG. 6 is a graphical representation of the air pressure supplied from the pump to the deep vein thrombosis prevention garment of the present invention, showing a maximum air pressure to be delivered, and the sequential pressure within the air filled chamber during an inflation cycle, before pressure supplied from the pump is released and all air filled chambers deflate; and

FIG. 7 is a top plan view of an alternative embodiment of the deep vein thrombosis prevention garment of the present invention showing a central panel having an air filled chamber with covering (with portions cut away for clarity), where the aft filled chamber includes a membrane panel and an aperture for releasing any over pressure.

### DETAILED DESCRIPTION OF A PREFERRED EMBODIMENT

Referring initially to FIG. 1, a top plan view of the preferred embodiment of the deep vein thrombosis prevention garment of the present invention is shown and generally designated 100. The deep vein thrombosis prevention garment 100 includes a single-layered body 102 having a central panel 104, a left side panel 106, and a right side panel 108. Garment panels 104, 106 and 108 are flexible and formed with a single layer. Single-layered body 102 has an inner surface 105 and an outer surface 103. In a preferred embodiment, the single layer of body 102 of the deep vein thrombosis prevention garment is made from durable cloth or other non-woven material that can comfortably contact a patients skin. For instance, a non-woven material SONTARA made from polyester and a substantially recyclable wood pulp may be suitable for use as the inside laminate for the device.

A flexible air supply tube 110 enters central panel 104 and leads to a single air filled chamber 111 (shown in dashed lines). This flexible air supply tube 110 having a non-descript length is shown. It is to be appreciated that the length of the air supply tube 110 may vary depending on the particular field of use, and the setting. For instance, in a hospital surgery setting, it may be difficult to position an air source immediately adjacent to the patient, and an extended air supply tube 110 is required.

Air is supplied to flexible air supply tube 110 from a pump 140. In a preferred embodiment, pump 140 includes a compressor capable of providing a predetermined maximum air pressure that provides a pressure force to fill the air filled chamber 111. Air supply tube 110 is equipped with a quick-disconnect connector 142 known in the industry to facilitate the changing of multiple deep vein thrombosis prevention garments 100 with pump 140. As will be described in greater detail below, pump 140 can provide air at a predetermined pressure for a predetermined period of time, providing for an inflation and deflation cycle according to the desired therapy parameters.

A cover panel 112 is sized to cover air filled chamber 111 and attached along its perimeter 114 to central panel 104. As shown, cover panel 112 may be heat fused to central panel 104 to seal air filled chamber 111 between the two (2) panels 112 and 104. Air filled chamber 111 may also be attached to central panel 104 to avoid movement in relation to body 102 during use.

Body 102, in a preferred embodiment, is made from a material that exhibits different amounts of elasticity depending on the direction of the tension being applied. More specifically, body 102 may expand in transverse direction 116, and in longitudinal direction 118. In one embodiment, elasticity in direction 116 may be greater than elasticity in direction 118, thereby allowing the body 102 to be snugly wrapped around a patient's limb that might have a taper or enlarged midsection, such as a muscular calf. The elasticity in direction 116 allows the body 102 to stretch to accommodate the limb. Alternatively, body 102 may have lithe or no elasticity in orthogonal direction 118. The combination of elasticity in a transverse direction 116 and lithe or no elasticity in longitudinal direction 118 provides for the deep vein thrombosis prevention garment 100 that is sufficiently structurally rigid to provide a compressive force when air filled chamber 111 is inflated, yet elastic enough to accommodate limbs having differing shapes.

As shown in FIG. 1, right side panel 108 and left side panel 106 are each formed with attachment straps 120 and 122, respectively, with each attachment strap having an integral fastener 126 and 128, respectively. In a preferred embodiment, straps 120 and 122 are provided with the hook portion of hook-and-loop style fasteners 126 and 128. This hook portion of the hook-and-loop fastener cooperates with the outside surface 103 of body 102 to allow the deep vein thrombosis prevention garment 100 of the present invention to be positioned about a patient's limb and secured in place by wrapping the panels 104, 106 and 108 around the limb, and pressing the fasteners 126 and 128 on straps 120 and 122 respectively, firmly against the outside surface 103 of panel 104. The hook-and-loop fasteners are attached to the outside surface 103 of panel 104 to hold the straps 120 and 122 in place. This type of fastener and method of attachment of the deep vein thrombosis prevention garment 100 provide a deep vein thrombosis prevention garment for patients having limbs of different sizes and can accommodate large or small diameter limbs simply by wrapping the panels 104 and 106 around the limb and securing straps 120 and 122 in place. In a preferred embodiment, polyester made from recycled bottles may be used for the loop fastener material, such as yarn made from recycled polyester available from UNIFI under the trade name REPREVE.

While the preferred embodiment of the deep vein thrombosis prevention garment of the present invention is manufactured having hook-and-loop type fasteners 126 and 128, it is to be appreciated that any other fastener known in the art may be used without departing from the present invention.

Referring now to FIG. 2, the deep vein thrombosis prevention garment 100 of the present invention is shown being used by a patient 200 for the prevention of deep vein thrombosis. Specifically, as shown, the deep vein thrombosis prevention garment 100 is positioned around the lower leg 202, or calf, of patient 200 and in communication with pump 140 through flexible air supply tube 110. Pump 140 supplies pressurized air through flexible air supply tube 110 to pressurize the air filled chamber 111 (shown in FIG. 1) within the deep vein thrombosis prevention garment 100.

FIG. 2 depicts a patient in a sitting position. However, this is merely exemplary of the typical use of the deep vein thrombosis prevention garment 100 of the present invention. Indeed, the deep vein thrombosis prevention garment of the present invention may be used with the patient in virtually any position. As will be described in greater detail below, the inflation and deflation cycle of the air filled chamber 111 may vary depending on the particular patient, and the particular environment. For instance, a patient using the deep vein thrombosis prevention garment of the present invention in a sitting position may opt for a faster inflation and deflation cycle, and may utilize higher air pressure in the air filed chamber 111 than a patient using the deep vein thrombosis prevention garment in a supine position on an operating table.

It is also to be appreciated that while FIG. 2 depicts a patient 200 having one (1) deep vein thrombosis prevention garment on a leg, a number of deep vein thrombosis prevention garments may be used simultaneously. For instance, in a surgery setting, it is commonplace to utilize the deep vein thrombosis prevention garment of the present invention on both legs.

As shown, the deep vein thrombosis prevention garment 100 is positioned around the calf 202 of patient 200 by positioning panel 104 against the patient's leg, and then wrapping straps 120 and 122 of panels 108 and 106, respectively, (shown in FIG. 1), around the calf 202 in opposite directions, and securing the straps to the outside surface 103 (shown in FIG. 1) of panel 104 with fasteners 126 and 128 (shown in FIG. 1).

Referring now to FIG. 3, a side cross-sectional view of the deep vein thrombosis prevention garment of the present invention as taken along line 3-3 of FIG. 1 is shown. As shown in FIG. 3, the construction of the deep vein thrombosis prevention garment 100 includes central panel 104 with air filled chamber 111 covered by cover panel 112. Cover panel 112 is joined to central panel 104 along the perimeter 114. In a preferred embodiment, bonds along the perimeter 114 may be made from heat bonds between the central panel 104 and cover panel 112, or cover panel 112 may be sewn, glued, or otherwise fastened as known in the art to central panel 104.

Air filled chamber 111 is made using two (2) sheets 134 and 136 which together define an air cavity 132. Sheets 134 and 136 are flexible and durable, and capable of withstanding prolonged periods of inflation and deflation without damage.

In a preferred embodiment, air chamber sheets 134 and 136 are made from ECO friendly polylactic acid (PLA) films that are made from either corn or sugar. PLA is an advanced type of packaging that has typically been used for application to the containers of soft drinks and dairy products; however, it can be a film overwrap and is heat-shrinkable to size. Alternatively, the panels may be made of petroleum-based plastic films such as Polyethylene terephthalate (PET), Oriented polystyrene (OPS) and Polyvinyl chloride (PVC). Nevertheless, the PLA shrink film that has been recently-developed is an eco-friendly alternative that is typically made of corn starch. Unlike petroleum-based films, the PLA film is naturally biodegradable and provides an environmentally conscious option.

One benefit of using sheeting 134 and 136 is the ability to create seals, such as seals 138 to form the air filled chamber 111. These seals 138 may be made by sonic welding, heat sealing, or any other method known in the art. It is important to note that chamber 111 is formed using two (2) sheets 134 and 136, and the seals 138 provide for an air-tight seal between the two (2) sheets 134 and 136 and allow for the pressurization of the resulting chamber. Specifically, sheets 134 and 136 are sealed together at seals 138 to establish a flexible, yet air-tight bond between the two (2) sheets 134 and 136.

Air supply tube 110 leads to an net 144 which extends into air cavity 132 sufficient that air supplied from pump 140 (shown in FIGS. 1-2) flows in direction 146 into air filled chamber 111 to inflate the chamber during use. Inlet 144 is sealed to sheet 136 to prevent leakage. For clarity, directional arrows 140 depict the typical airflow from the flexible air supply tube 110 to the air filled chamber 111. From this Figure, the expandability of the air filled chamber 111 is easily appreciated. As air filled chamber 111 is provided with pressurized air from tube 110 and pump 140, the pressure in the air filled chamber 111 will continue to rise until the air filled chamber pressure equalizes with the pressure of the air from pump 140.

Referring now to FIGS. 4 and 5, the deep vein thrombosis prevention garment 100 of the present invention is shown as used on the leg 202 of a patient 200 (shown in FIG. 2) starting with an un-inflated configuration in FIG. 4, and the inflated configuration in FIG. 5.

Starting with FIG. 4, an exemplary cross-sectional view of the deep vein thrombosis prevention garment 100 of the present invention as used on the leg 202 of the patient 200, depicting the deep vein thrombosis prevention garment 100 in a deflated configuration, is shown. In the deflated configuration, little or no pressure is exerted on the leg 202 of the patient and blood flows unrestrictedly through the leg. As air is introduced into tube 110 (shown in FIGS. 1-3) and begins to fill air cavity 132 of air filled chamber 111 (as depicted in FIG. 3 with air flows 146), pressure is introduced to leg 202.

As shown in FIG. 5, as air is continually introduced into air cavity 132 of air filled chamber 111 (shown in FIGS. 1 and 3), an increase in pressure is applied to leg 202 of the patient to urge blood flow upward from the foot 204 through the leg 202 in direction 206. Specifically, inflation of air filled chamber 111 creates a pressure on the limb 202 of patient 200.

When the inflation cycle is completed, the air pump 140 (shown in FIGS. 1-2) releases the air pressure to air supply tube 110, and air dissipates through tube 110 in reverse of direction 146 (shown in FIG. 3) to return the deep vein thrombosis prevention garment 100 of the present invention to its deflated state as shown in FIG. 4. Following a delay, this cycle is repeated according to a particular patient profile, and may be repeated for extended periods of time in order to minimize the likelihood that thrombosis will develop in the patient.

This cyclic pressure of an inflation cycle, in combination with the inter-venous valves present in the circulatory system, provides a peristaltic force on blood within the limb. The peristaltic force results in the near continual movement of blood within the limb being treated, thereby avoiding the formation of deep vein thrombosis.

Referring now to FIG. 6, a graphical representation of the air pressure supplied from the pump 140 (shown in FIGS. 1-2) to the deep vein thrombosis prevention garment 100 of the present invention is shown and generally designated 250. Graph 250 includes a vertical Air Pressure axis and a horizontal Time axis. This graph 260 depicts a typical inflation and deflation cycle that occurs in the deep vein thrombosis prevention garment of the present invention.

Graph 250 includes a primary supply air pressure curve 252 which corresponds to the air provided by pump 140 to flexible air supply tube 110 (shown in FIGS. 1 and 3). This air supply begins at the start of the inflation cycle and rises to a maximum supplied air pressure 254. This maximum supplied air pressure 254 is approximately equal to an overall maximum pressure 256 (shown by dashed line) that corresponds to the maximum desired pressure within air filled chamber 111 (shown in FIGS. 1 and 3), the maximum pressure medically safe, or any other maximum value utilized in the art to ensure safe operation of the deep vein thrombosis prevention garment 100.

In the deep vein thrombosis prevention garment of the present invention, the preferred maximum pressure for air filled chamber 111 is 35 mmHg. It is to be appreciated, however, that different air pressures may be utilized for differing applications, treatment positions, duration of treatment, and other factors known and considered in the art.

The inflation cycle is completed once the air filled chamber 111 has had sufficient time to inflate. Following the inflation cycle, a delay 258 may be utilized to maintain a constant pressure on the limb 202 (shown in FIGS. 2 and 4-5) to provide time for the blood to flow through the limb. Following any delay, the deflation cycle begins and the pressure 260 in air supply tube 110 decreases to zero.

As the decrease in air supply tube pressure 260 occurs, the pressure 262 in air filled chamber 111 likewise returns to zero in substantially the same time. Once this inflation and deflation cycle is complete, a delay 264 may be inserted prior to beginning the next inflation and deflation cycle.

Using the deep vein thrombosis prevention garment 100 of the present invention, the time for a complete inflation cycle, deflation cycle and delay is approximately twenty (20) seconds. As a result, the deep vein thrombosis prevention garment 100 can be cycled three (3) times every minute in order to provide a continuous force to create the desired peristaltic effect. It is to be appreciated that the specific period for a complete cycle may be changed depending on the size of the limb being treated, the pressure desired, and the peristaltic forces necessary to minimize the likelihood of the development of a thrombosis.

### Alternative Embodiments

Referring now to FIG. 7, an alternative embodiment of the deep vein thrombosis prevention garment of the present invention is shown and generally designated 300. Garment 300 includes central panel 304 having an air filled chamber 311 shown with covering 310 (with portions cut away for clarity). Air filled chamber 311 includes a membrane panel 320 for releasing any over-pressure. Specifically, an aperture 322 may be formed in air filled chamber 311 and covered with membrane panel 320. Membrane panel 320 is a non-woven material that provides resistance to the flow of air through the membrane. When the pressure within chamber 311 exceeds a maximum value, air passes, through membrane 320 to release the excess pressure thereby preventing excessive air pressure within the air filled chamber 311.

Membrane panel 320 may be affixed to sheet 324 of air filled chamber 311 using heat sealing, sonic welding, adhesive, or other methods known in the art. Membrane panel 320 is sized larger than aperture 322 and may be selected from synthetic non-woven materials having varying air transmissivity ratings thereby allowing the pressure within air filled chamber 311 to be regulated to a maximum value.

While there have been shown what are presently considered to be preferred embodiments of the present invention, it will be apparent to those skilled in the art that various changes and modifications can be made herein without departing from the scope and spirit of the invention.

1. A deep vein thrombosis prevention garment, comprising:
a central panel having an air chamber receiving air from a flexible air supply tube and an outer surface;
a cover having a perimeter and positioned over said air chamber to capture said air chamber between said central panel and said cover;
a left side panel extending from a lower portion of said central panel formed with one or more attachment straps having an integral fastener attachable to said central panel; and
a right side panel extending from an upper portion of said central panel and formed with one or more attachment straps having an integral fastener attachable to said central panel,
wherein the integral fastener of the left side panel attaches to a lower portion of the outer surface of the central panel and the integral fastener of the right side panel attaches to an upper portion of the outer surface of the central panel.

2. The deep vein thrombosis prevention garment of claim 1, further comprising:
an aperture formed in said air chamber; and
a membrane panel covering said aperture and configured to pass air having a predetermined minimum pressure from said air chamber.

3. The deep vein thrombosis prevention garment of claim 2, further comprising said predetermined minimum pressure being in the range from 25 mmHg to 35 mmHg.

4. The deep vein thrombosis prevention garment of claim 1, further comprising said predetermined minimum pressure being 25 mmHg.

5. The deep vein thrombosis prevention garment of claim 1, further comprising a pump in communication with said flexible air supply tube to provide air having a predetermined pressure sufficient to inflate said air chamber.

6. The deep vein thrombosis prevention garment of claim 5, wherein said predetermined pressure is 35 mmHg.

7. The deep vein thrombosis prevention garment of claim 5, further comprising said pump configured to provide air at the predetermined pressure for a fixed period of time.

8. The deep vein thrombosis prevention garment of claim 1, further comprising a means for releasing air from said air chamber if said air exceeds a pressure of 35 mmHg.

9. The deep vein thrombosis prevention garment of claim 1, further comprising a means for pressurizing said air chamber to 35 mmHg.

10. The deep vein thrombosis prevention garment of claim 1, wherein the garment is constructed from an elastic material.

11. The deep vein thrombosis prevention garment of claim 10, wherein the elastic material has greater elasticity in a lateral direction and less elasticity in a vertical direction.

12. The deep vein thrombosis prevention garment of claim 1, wherein the integral fastener is a hook and loop type fastener.

13. The deep vein thrombosis prevention garment of claim 12, wherein the cover is the loop portion of the hook and loop type fastener.

14. The deep vein thrombosis prevention garment of claim 1, wherein the cover is bonded to the central panel along the perimeter of the central panel.

15. The deep vein thrombosis prevention garment of claim 14, further comprising a means to bond the cover to the central panel.

16. The deep vein thrombosis prevention garment of claim 1, wherein the air chamber is constructed from two (2) sheets polylactic acid film, polyethylene terephthalate, oriented polystyrene, or polyvinyl chloride.

17. The deep vein thrombosis prevention garment of claim 1, wherein the air chamber is constructed from biodegradable material.

18. The deep vein thrombosis prevention garment of claim 2, wherein the membrane panel is attached to the air chamber using heat sealing, sonic welding, or adhesive.
